SEQUENCE LISTING (7 sequences)

SEQ ID NO: 1 - DNA, 20 bp, Unknown
  glutathione transferase p (GSTp) multiplex primer
ctcaaaaggcttcagttgcc

SEQ ID NO: 2 - DNA, 20 bp, Unknown
  glutathione transferase p (GSTp) multiplex primer
ggagcaagcagaggagaatc

SEQ ID NO: 3 - DNA, 20 bp, Unknown
  glutathione transferase p (GSTp) multiplex primer
aaggatggacaggcagaatg

SEQ ID NO: 4 - DNA, 20 bp, Unknown
  glutathione transferase p (GSTp) multiplex primer
ggctgtgtctgaatgtgagg

SEQ ID NO: 5 - DNA, 19 bp, Unknown
  glutathione transferase p (GSTp) multiplex primer
cgaaggccttgaacccact

SEQ ID NO: 6 - DNA, 20 bp, Unknown
  glutathione transferase p (GSTp) multiplex primer
cgtgtgtgtgtgtacgcttg

SEQ ID NO: 7 - DNA, 20 bp, Unknown
  glutathione transferase p (GSTp) multiplex primer
cagacacagagcacatttgg

(19) United States
(12) Patent Application Publication
(10) Pub. No.: US 2012/0003710 A1
(43) Pub. Date: Jan. 5, 2012

(54) Isolation of Biomolecules from Biological Samples

(76) Inventors: Barbara Dawn Leinweber, Tucson, AZ (US); Paul E. Diggins, Tucson, AZ (US)

(21) Appl. No.: 13/031,599

(22) Filed: Feb. 21, 2011

Related U.S. Application Data
(60) Provisional application No. 61/306,447, filed on Feb. 19, 2010.

(51) Int. Cl. C12N 11/14; C07H 21/02; B65D 71/00; C07H 21/04; C07K 17/14; B82Y 99/00; B82Y 5/00

(52) U.S. Cl. 435/176; 536/23.1; 530/402; 206/223; 977/773; 977/904

(57) ABSTRACT

Nanoparticles for use in the collection, concentration, isolation and storage of biomolecules from biological samples are provided. More specifically, nanoparticles used to isolate biomolecules, including nucleic acids and proteins, cells, cell fragments, bacteria, and viruses from biological samples such as urine, cerebrospinal fluid (CSF), mouthwash samples, and amniotic fluid are provided. Kits for using nanoparticles for the isolation of biomolecules are also provided.

### CROSS-REFERENCE TO RELATED APPLICATIONS

This application claims priority to U.S. Provisional Patent Application No. 61/306,447, for Isolation of Cell Free DNA using Nanoparticles, filed on 19 Feb. 2010, which is incorporated herein in its entirety.

### FIELD

Nanoparticles for use in the isolation and storage of biomolecules from biological samples are provided.

### BACKGROUND

Biological materials of interest are often found in biological samples from which it is difficult to isolate or purify those biological materials. One such example is the isolation and purification of nucleic acids and proteins from urine which has been particularly challenging due to the high concentration of salts, nucleases, and proteases in urine. The salts, nucleases, and other inhibitors present in urine make it difficult to isolate the biomolecules of interest and also tend to interfere with downstream applications using the biomolecules.

DNA in urine can generally be divided into two fractions. When urine is subjected to sedimentation at a few hundred to a few thousand times the force of gravity, the sediment contains cells and debris. The cell free DNA, termed transrenal DNA (trDNA) remains in the supernatant. In order to be present in urine, DNA may be derived from the urinary tract or may filter into the urine from the general circulation. DNA from pathogens, DNA associated with various cancers, donor DNA from transplant patients, and transrenal fetal DNA have been detected in urine.

### SUMMARY

Nanoparticles can be used for the collection, concentration, isolation and storage of biomolecules from biological samples. More specifically, nanoparticles can be used to isolate biomolecules, including nucleic acids and proteins, cells, cell fragments, bacteria, and viruses from biological samples such as urine, cerebrospinal fluid (CSF), mouthwash samples, and amniotic fluid. Nanoparticles can be used to separate out the biomolecules of interest from biological samples that contain components that would otherwise degrade or inhibit detection of the biomolecules of interest.

In one embodiment, nanoparticles are used for the isolation and storage of nucleic acids and protein from urine. Nucleic acids and associated biomolecules are separated from the bulk of urine salts by their ability to bind to oxyanion passivated mineral, metal oxide, or composite metal oxide nanoparticles. Nucleases are inhibited simultaneously with the digestion of proteins that co-isolate in a solution that contains a pH buffer, β-mercaptoethanol (β-ME), and guanidine HCl. Savinase is a preferred protease because it lacks disulfide bonds that could be reduced by β-ME. The pH and concentration of β-ME, and guanidine HCl can be changed for alternate proteases.

In another embodiment, nanoparticles may be used for the isolation of biomolecules from samples such as urine in locations where a centrifuge or electricity may not be available. In this embodiment, the nanoparticles preferably are able to settle out of aqueous solution without the use of centrifugation. When the biological materials that are isolated with these nanoparticles are desiccated, those biological materials, such as DNA, RNA, and proteins, can be stabilized for extended periods of time. The biological molecules can then be stored or sent to different location for further analysis.

### BRIEF DESCRIPTION OF THE DRAWINGS

FIG. 1 is a photograph of an agarose gel with DNA isolated from urine using passivated kaolin (mineral) particles, zirconia (metal oxide) particles, and yttria-stabilized zirconia (composite metal oxide) particles.

FIG. 1A is a photograph of an agarose gel showing DNA isolated from urine using 730×g centrifugation in the presence and absence of kaolin particles.

FIG. 1B is a photograph of an agarose gel showing DNA isolated from a single batch of urine using kaolin particles, zirconia particles, and no particles (control, C) using 730×g centrifugation.

FIG. 1C is a photograph of an agarose gel showing DNA isolated from three different urine collections using yttria-stabilized zirconia (YSZ) or no particles (control, C) using 730×g centrifugation.

FIG. 2 is a photograph of an agarose gel showing the PCR products resulting from multiplexed GST1p PCR using the samples from FIG. 1. The expected sizes of the amplicons are 167, 244, 340, 397, 473, and 551 base pairs.

FIG. 3 shows that borate-passivated yttria-stabilized zirconia (YSZ-b) particles allow for the isolation of DNA from urine using separation by gravity.

FIG. 3A is a graph comparing DNA yields using borate-passivated yttria-stabilized zirconia (YSZ-b) particles and comparing 730×g centrifugation and 1×g settling.

FIG. 3B is a photograph of an agarose gel showing the DNA from FIG. 3A resolved on a 2.2% agarose gel.

FIG. 3C is a photograph of an agarose gel showing the PCR products resulting from multiplexed GST1p PCR using the samples from FIG. 3A. Expected sizes of amplicons are 167, 244, 330, 397, 473, and 551 base pairs.

FIG. 4 shows how the volume of borate-passivated zirconia (ZrO₂-b) particles and borate-passivated yttria-stabilized zirconia (YSZ-b) particles used affects the quantity and quality of DNA isolated from various urine samples.

FIG. 4A shows four different urine collections used to examine the influence of the volume of borate-passivated zirconia (ZrO₂-b) particles and borate-passivated yttria-stabilized zirconia (YSZ-b) particles on the amount and type of DNA isolated. The hollow symbols represent two collections of urine using the borate-passivated yttria-stabilized zirconia (YSZ-b) particles. The filled symbols represent two separate collections of urine processed with the borate-passivated zirconia (ZrO₂-b) particles. The black circles represent a sample that was processed directly, and the gray circles represent a sample that was centrifuged at 4000×g for 15 minutes before processing.

FIG. 4B is a photograph of an agarose gel showing DNA isolated from urine with no pre-centrifugation step to remove cell debris using an increasing volume of borate-passivated zirconia (ZrO₂-b) particles. This sample corresponds to the black circles in FIG. 4A.

FIG. 4C is a photograph of an agarose gel showing DNA isolated from urine that had been centrifuged at 4000×g for 15 minutes to prior to treatment with an increasing volume of borate-passivated zirconia (ZrO₂-b) particles. DNA from the pellets appears is in lanes 2 and 3, labeled P1, and P2. DNA extracted from the supernatant with borate-passivated zirconia (ZrO₂-b) particles is in the remaining lanes. In order to visualize the DNA, the image has been enhanced through Adobe Photoshop.

FIG. 5 shows the influence of the incubation time with borate-passivated yttria-stabilized zirconia (YSZ-b) particles on the amount and quality of DNA yielded.

FIG. 5A is a graph showing the amount of DNA recovered from two 50 ml aliquots (first batch and second batch) of a single urine collection as a function of time incubating the urine with borate-passivated yttria-stabilized zirconia (YSZ-b) particles before 730×g centrifugation.

FIG. 5B is a photograph of an agarose gel showing the DNA isolated at various time points from the urine collections of FIG. 5A. The arrows indicate increasing incubation times with borate-passivated yttria-stabilized zirconia (YSZ-b) particles before DNA isolation.

FIG. 5C is a photograph of an agarose gel showing the PCR products resulting from multiplexed GST1p PCR using the samples from FIG. 5A. The arrows indicate increasing incubation times with borate-passivated yttria-stabilized zirconia (YSZ-b) particles before DNA isolation. Expected sizes of amplicons are 167, 244, 330, 397, 473, and 551 base pairs.

FIG. 6 shows the differences in DNA obtained from urine using fluoride/phosphate passivated kaolin, borate-passivated yttria-stabilized zirconia (YSZ-b), and borate-passivated zirconia (ZrO₂-b) particles. For each of the three particles types, there are three methods (labeled 1 through 3) for treating the samples. Each sample was centrifuged at 730×g for 5 minutes yielding a pellet and a supernatant. Method 1 (FIG. 6A lanes 2, 5, and 9; FIG. 6B lanes 3, 6, and 11) contains DNA from the pellet with no particles. Method 2 (FIG. 6A lanes 3, 6, and 10; FIG. 6B lanes 4, 7, and 12) contains DNA from the pellet subsequently treated with particles. Method 3 (FIG. 6A lanes 4, 7, and 11; FIG. 6B lanes 5, 8, and 13) contains DNA from the supernatant subsequently treated with particles.

FIG. 6A is a photograph of an agarose gel showing the DNA isolated with each of the three particles types using each of the three methods.

FIG. 6B is a photograph of an agarose gel showing the PCR products resulting from multiplexed GST1p PCR using the samples from FIG. 6A. Expected sizes of amplicons are 167, 244, 330, 397, 473, and 551 base pairs.

FIG. 7 shows a comparison between DNA isolated from urine using a 2-propanol DNA extraction process (labeled 2PrOH precip) and DNA isolated from urine using borate-passivated zirconia (ZrO₂-b) particles (labeled ZrO2-b).

FIG. 7A shows UV absorption spectra for DNA isolated from urine using a 2-propanol DNA extraction process (black lines) and for DNA isolated from urine using borate-passivated zirconia (ZrO₂-b) particles (dotted lines).

FIG. 7B is a photograph of an agarose gel showing the isolated DNA from both methods resolved on a 2.2% agarose gel. DNA isolated using borate-passivated zirconia (ZrO₂-b) particles was rerun on a separate gel (FIG. 7B, right panel).

FIG. 7C is a photograph of an agarose gel showing the PCR products resulting from multiplexed GST1p PCR using the samples from FIG. 7B. Expected sizes of amplicons are 167, 244, 330, 397, 473, and 551 base pairs.

FIG. 8 shows the isolation of exogenous lambda (λ) bacteriophage DNA that was added to urine using borate-passivated yttria-stabilized zirconia (YSZ-b) particles.

FIG. 8A is a graph showing the amount of DNA recovered as a function of the amount of exogenous lambda (λ) DNA added to the sample.

FIG. 8B is a photograph of an agarose gel showing the DNA isolated from samples in Example 14 to which exogenous lambda (λ) DNA was added.

FIG. 9 is a photograph of an agarose gel showing DNA isolated from urine from a chemotherapy patient before and after chemotherapy treatment using borate-passivated yttria-stabilized zirconia (YSZ-b) particles.

FIG. 9A is a photograph of an agarose gel showing DNA isolated before treatment (labeled B, lane 2) and after one (labeled 1, lane 3) and two (labeled 2, lane 4) doses of the chemotherapeutic agent erlotinib.

FIG. 9B is a photograph of an agarose gel showing the PCR products resulting from multiplexed GST1p PCR using the samples from Example 15 and FIG. 9A. Expected sizes of amplicons are 167, 244, 330, 397, 473, and 551 base pairs.

FIG. 10 shows DNA isolated from urine after about two weeks of storage at 55° C., simulating long-term storage for approximately eight weeks.

FIG. 10A is a photograph of an agarose gel showing DNA isolated from urine after simulated simulating long-term storage.

FIG. 10B is a photograph of an agarose gel showing the PCR products resulting from multiplexed GST1p PCR using the samples from Example 16 and FIG. 10A. Expected sizes of amplicons are 167, 244, 330, 397, 473, and 551 base pairs.

FIG. 11 is a series of graphs of the UV absorbance of nucleic acids isolated from urine comparing borate passivated zirconia and phosphate passivated zirconia nanoparticles and three different elution buffers.

FIG. 12 relates to the detection of RNA in urine samples.

FIG. 12A is a UV spectrum from pooled human urine as described in Example 18.

FIG. 12B is an image of an RNA microarray slide from an RNA analysis performed by High Throughput Genomics, Tucson, Ariz., on RNA isolated from urine.

FIG. 13 is a table showing the results of an mRNA microarray analysis of the nucleic acids isolated from urine for a series of mRNA house keeping genes.

FIG. 14 is a table showing the results of an miRNA microarray analysis of the nucleic acids isolated from urine.

### DETAILED DESCRIPTION OF THE PREFERRED EMBODIMENTS

Nanoparticles for use in the isolation and storage of biomolecules from biological samples are provided. Often, the biological samples in which biomolecules of interest are found also contain components that cause the degradation of the biomolecules or otherwise interfere with the detection and isolation of the biomolecules of interest. Biological fluids often contain high concentrations of salts, nucleases, and other inhibitors. By using appropriately chosen nanoparticles, DNA, RNA, proteins, cells, cell fragments, bacteria, viruses, and other biomolecules of interest can be purified and isolated from biological fluids and cell culture medium. Nanoparticles can be used for the collection, concentration, isolation and storage of biomolecules including nucleic acids and proteins from biological samples such as cerebrospinal fluid (CSF), mouthwash samples, urine, and amniotic fluid. In another embodiment, the biological sample is generated by washing fruits and vegetables with a soapy water solution. This type of sample can be used to detect bacterial and other types of contamination that may be present. In various preferred embodiments, the biological samples have a low protein content and are of low viscosity.

One example is the isolation and purification of nucleic acids and proteins from urine. Isolation and purification of biomolecules from urine has been particularly challenging due to the high concentration of salts, nucleases, and proteases in urine. The salts, nucleases, and other inhibitors present in urine tend to be detrimental both to the detection and isolation of biomolecules in the urine and to downstream applications using those biomolecules, such as PCR using DNA and RNA from urine.

Preferred nanoparticles have a density and a sedimentation velocity that allows them to maintain a colloidal suspension in solution for long enough to interact with the biomolecules of interest in a biological sample. In one preferred embodiment, the nanoparticles settle out of suspension from a liquid sample through gravity without the use of electrical centrifugation. This allows the use of the nanoparticles for isolation of biomolecules in areas where electricity or centrifugation facilities may not be available. In one embodiment, about 90% of the nanoparticles settle out of suspension from an aqueous solution within between about 15 minutes and about 4 hours. In one embodiment, about 90% of the nanoparticles settle out of suspension from an aqueous solution within between about 30 minutes and about 4 hours. In one embodiment, about 90% of the nanoparticles settle out of suspension from an aqueous solution within between about 30 minutes and about 3 hours. In one embodiment, about 90% of the nanoparticles settle out of suspension from an aqueous solution within between about 30 minutes and about 2 hours. In one embodiment, about 90% of the nanoparticles settle out of suspension from an aqueous solution within between about 30 minutes and about 1 hour. In one embodiment, about 90% of the nanoparticles settle out of suspension from an aqueous solution within between about 1 hour and about 2 hours. In one embodiment, about 90% of the nanoparticles settle out of suspension from an aqueous solution within between about 1 hour and about 3 hours. In one embodiment, about 90% of the nanoparticles settle out of suspension from an aqueous solution within between about 1 hour and about 4 hours.

Preferably, the nanoparticles are stable under the conditions required by the biological sample of interest. For example, urine is generally acidic at about pH 5, and nanoparticles used for the isolation of biomolecules from urine are preferably stable at pH 5. For nanoparticles that undergo passivation, the nanoparticles are preferably stable at an acid pH required to carry out the passivation process. In one embodiment, the nanoparticles are stable at an acidic pH as low as about pH 5. In one embodiment, the nanoparticles are stable at an acidic pH as low as about pH 4. In one embodiment, the nanoparticles are stable at an acidic pH as low as about pH 3. In one embodiment, the nanoparticles are stable at an acidic pH as low as about pH 2. In one embodiment, the nanoparticles are stable at an acidic pH as low as about pH 1.

Nanoparticles may comprise a metal oxide, a composite metal oxide, or a metal oxide containing mineral. In one preferred embodiment, the nanoparticles comprise an oxyanion and a halide passivation agent with a metal oxide, a composite metal oxide, or a metal oxide containing mineral.

Examples of nanoparticles include, but are not limited to, a fluoride/phosphate passivated metal oxide containing mineral such as kaolin; a borate (Na₂B₄O₇) passivated pure metal oxide such as zirconia (zirconium oxide, ZrO₂); and a borate (Na₂B₄O₇) passivated composite metal oxide such as yttria-stabilized zirconia (YSZ). Other examples include cerium oxide (CeO₂); magnesium oxide (MgO); neodymium oxide (Nd₂O₃); tungsten (VI) oxide (WO₃), and aluminum oxide (Al₂O₃) that may be passivated or unpassivated. Preferred passivating agents include, but are not limited to, phosphate, borate, fluoride, sulfate, and bromide.

Among the preferred nanoparticles are the following: kaolin (Engelhard-BASF, ASP Ultrafine) of approximately 200 nm diameter; zirconia (zirconium oxide, ZrO₂) having a particle size of approximately less than 100 nm, a surface area of about greater than or equal to 25 m²/g, and a density of about 5.89 g/mL at 25° C.; and yttria-stabilized zirconia. Some of the materials useful for nanoparticles are shown in Table 1 with their densities. Nanoparticles preferably have a density greater than about 2. In one embodiment, the nanoparticles have a density between about 2 and about 9. In another embodiment, the nanoparticles have a density between about 2 and about 8. In another embodiment, the nanoparticles have a density between about 2 and about 7.5. In another embodiment, the nanoparticles have a density between about 2.5 and about 7.5. In another embodiment, the nanoparticles have a density between about 3 and about 7.5. In another embodiment, the nanoparticles have a density between about 3.5 and about 7.5. In another embodiment, the nanoparticles have a density between about 4 and about 7.5. In another embodiment, the nanoparticles have a density between about 2 and about 7.5. In another embodiment, the nanoparticles have a density between about 5.5 and about 6.5. In another embodiment, the nanoparticles have a density between about 5.5 and about 7.5.

TABLE 1

| Material | Density (g/cm³) |
|---|---|
| Kaolin | 2.6 |
| Aluminum oxide (Al₂O₃) | 2.7-2.9 |
| Magnesium oxide (MgO) | 3.6 |
| Zirconium Oxide (ZrO₂) | 5.9 |
| Yttria Stabilized Zirconia (YSZ) | 5.9-6.1 |
| Nickel Oxide (NiO) | 6.7 |
| Cerium Oxide (CeO₂) | 7.1 |
| Neodymium Oxide (Nd₂O₃) | 7.2 |
| Tungsten Oxide (WO₃) | 7.2 |

In one embodiment, the use of nanoparticles allows for the isolation of biomolecules from samples such as urine. When the isolated biological materials are desiccated, those biological materials, such as DNA, RNA, and proteins, can be stabilized for extended periods of time. The biological molecules can then be stored or sent to a different location for further analysis.

In one embodiment, zirconium oxide (ZrO₂, or zirconia) based nanoparticles are used for the isolation and storage of nucleic acids and protein from urine. In another embodiment, nanoparticles are used for the isolation and storage of nucleic acids and protein from urine in locations where a centrifuge or electricity may not be available. The nucleic acids and associated biomolecules are separated from the bulk of urine salts by their ability to bind to oxyanion passivated mineral, metal oxide, or composite metal oxide nanoparticles. Nucleases are inhibited simultaneously with the digestion of proteins that co-isolate in a solution that contains a pH buffer, β-mercaptoethanol (β-ME), and guanidine HCl. Savinase is a preferred protease because it lacks disulfide bonds that could be reduced by β-ME.

In various embodiments, the nanoparticles are passivated with an oxyanion of boron, phosphorous, sulfur, or other elements in these periodic groups of elements. Halides such as fluoride may also be used in conjunction with the aforementioned oxyanions for passivation.

In various embodiments, the nanoparticles may comprise a metal oxide mineral such as kaolin (a silica aluminum oxide, Al₂Si₂O₅(OH)₄), corundum (Al₂O₃), cassiterite (SnO₂), or any of the spinel class of minerals known for their hardness and high specific gravity. The spinels are any of a class of minerals of general formulation A2+B23+O42− which crystallize in the cubic (isometric) crystal system, with the oxide anions arranged in a cubic close-packed lattice with the cations A and B occupying some or all of the octahedral and tetrahedral sites in the lattice. A and B can be divalent, trivalent, or quadrivalent cations, including magnesium, zinc, iron, manganese, aluminum, chromium, titanium, and silicon. Although the anion is normally oxide, structures are also known for the rest of the chalcogenides. A and B can also be the same metal under different charges, such as the case in Fe3O4 (as Fe2+Fe23+O42−). The nanoparticles may comprise a metal oxide such as, for example, zirconia, ceria, aluminum oxide, or titanium dioxide. The nanoparticles may also comprise composite metal oxides such as, for example, yttria stabilized zirconia, europium doped zirconia, or titanium doped zirconia.

In various embodiments, kits and instructions for using kits containing nanoparticles for the isolation of biomolecules are also provided.

General Procedures

In one embodiment, the procedure is generally carried out as follows. A biological fluid, such as urine, is collected into a vessel with a nanoparticle suspension and is mixed by shaking. The biomolecules within the sample aggregate onto the nanoparticles. The nanoparticles can settle by sedimentation or by low speed mechanical centrifugation. The biological fluid is decanted. At this point, if storage or field transport is desired, 15 to 20 mL of 70% 2-propanol is added to the pellet and mixed. Once the pellet is settled by centrifugation or by gravity, the excess alcohol is decanted and the pellet is air dried. When the biomolecules are bound to the nanoparticles, they are stabilized and resistant to degradation and can be subject to short- or long-term storage and transport. Alcohol is used to dehydrate and wash the pellet of DNA and nanoparticles. The sample can be transported to a secondary location or directly processed to further purify the DNA. For DNA isolation, the biomolecule/nanoparticle matrix is digested with 0.25 U Savinase per mL in a solution containing 4 M guanidine HCl, 1× extraction buffer, and 2.5% (v/v) β-mercaptoethanol (β-ME). The 20× extraction buffer contains 10 mM CAPS (sodium salt (3-[cyclohexylamino]-1-propanesulfonic acid, sodium salt); 10 mM Na₂-CO₃; 10 mM EDTA; 100 mM NaCl; and 0.01% sodium lauroyl sarcosine. 70% 2-propanol is then added to cause the nucleic acids to precipitate onto the nanoparticles. The nanoparticles and nucleic acids settle into a pellet by gravity or centrifugation. The alcohol containing the proteins is decanted. If protein analysis is desired the alcohol can be concentrated and the proteins analyzed. The DNA bound to the nanoparticles can be stored or eluted and concentrated for further analysis.

### Example 1

### Preparation of Phosphate/Fluoride Treated Kaolin Particles

Phosphate/fluoride treated kaolin particles were prepared by suspending the kaolin nanoparticles ((Engelhard-BASF, ASP Ultrafine, CAS No. 1332-58-7) in deionized water at a weight to volume ratio of 1 to 3. This colloidal suspension was incubated for a minimum of 16 hours. The nanoparticles were washed by a sedimentation-resuspension process by first sedimenting the nanoparticles out of suspension by centrifugation at 4000×g for 10 minutes. Then the kaolin nanoparticles were resuspended in water at the same ratio. This process was repeated until the supernatant was clear with no sign of opalescence. The final kaolin pellet was resuspended at a weight to volume ratio of 1 to 3 with water, and an equal volume of 10% sulfuric acid was added to the suspension. This sulfuric acid/kaolin slurry was mixed and incubated at room temperature for 1 to 2 hours. Then the slurry was washed with distilled water by the sedimentation-resuspension process until the pH of the supernatant was the same as the pH of the distilled water. To this suspension, as a 1 to 10 ratio of weight of particles to volume of suspension, 1/50th volume of 500 mM NaF, to a final NaF concentration of approximately 10 mM was added. This suspension was mixed and then subjected to one round of sedimentation-resuspension with distilled water, with the pellet being resuspended in 100 mM NaH₂PO₄ at a ratio of 1 to 10 mixed for at least 16 hours. This suspension was subjected to three rounds of the sedimentation-resuspension with 1 mM NaH₂PO₄. The particle suspension was stored as this solution until ready for dilution in 1 mM NaH₂PO₄ and 10 mM NaF. The final suspension of kaolin nanoparticles was 100 mg to 200 mg particles/mL in a suspension solution of 1 mM NaH₂PO₄ and 10 mM NaF.

### Example 2

### Preparation of Borate-Passivated Zirconia (ZrO₂-b) Particles

100 mL of 1N sulfuric acid is added to 25 grams of ZrO₂. The particles are centrifuged at 6000 RMP for 10 minutes, and the supernatant is discarded. 200 mL of 10 mM Na₂B₄O₇ is added, and the particles are shaken for 15 minutes. The particles are then centrifuged at 6000 RMP for 10 minutes, the supernatant is discarded. Addition of Na₂B₄O₇, centrifugation, and discarding of the supernatant are repeat two additional times with the particles finally resuspended in 200 mL 10 mM Na₂B₄O₇.

### Example 3

### Preparation of Borate-Passivated Yttria-Stabilized Zirconia (YSZ-B) Particles

10 grams of ZrO₂ yttria particles are added to each of two containers. 10 ml of 20% sulfuric acid are added to each container. The particles are mixed by inversion and then tumbled for 1 hour at room temperature. The particles are centrifuged at 6000 RPM for 10 minutes, and the 20% sulfuric acid supernatant is discarded. 10 ml of 200 mM NaCl is added to each pellet, and the containers are placed in a paint shaker for 15 minutes. 9 ml of water is added to each container, and the containers are placed in a paint shaker for an additional 15 minutes. At this point, the pH of the supernatant is measured, and the pH is preferably between about pH 1 and about pH 1.5. The supernatant is decanted. 20 ml of 200 mM NaCl is added to each container, and the containers are placed in a paint shaker for 15 minutes. 20 mL of 200 mM NaCl is added to each container, and the containers are mixed by inversion. The containers are centrifuged at 6000×g for 10 minutes, and the supernatant is decanted. Add 12 ml of 100 mM borate is added to each container, and the containers are mixed by inversion or in a paint shaker, as required to resuspend the pellet. The containers are centrifuged at 6000×g for 10 minutes, and the supernatant is decanted. 12 ml of 10 mM borate is added to each container, and the containers are mixed by inversion. The containers are then tumble mixed for one hour at room temperature. The containers are centrifuged at 6000×g for 10 minutes, and the supernatant is decanted. Each 10 grams of pellet is resuspended in 10 mM Na₂B₄O₇.

### Example 4

### Sample Collection

Protocols were chosen to closely mimic conditions that might exist at point of care (P.O.C.) collection sites or at home collections. Urine from females was collected in 1 liter polypropylene beakers. Urine from males was collected in 1 liter polypropylene bottles. Urine was collected from two males and three females. Urine in 50 ml aliquots was distributed equally in polypropylene conical tubes already containing 0.5 ml borate-passivated yttria-stabilized zirconia (YSZ-b) particles, borate-passivated zirconia (ZrO₂-b) particles, or kaolin particles, as indicated. For point of care collection sites where no centrifugation would occur, borate-passivated yttria-stabilized zirconia (YSZ-b) particles are used because their density allows for sedimentation through gravity without centrifugation.

The concentration of particles was measured by optical density at 400 nm. The number was derived by diluting the sample 100 fold, such that the absorbance was between 0.1 and 1.0 units. An absorbance of 1.0 indicates that the particle density is sufficient to scatter 90% of the incident light (JO.

A=−log₁₀(I/I₀)

Working stock particle densities ranged from 45 to 98 absorbance units. Different varieties of particles were compared based on optical density at 400 nm. Borate-passivated yttria-stabilized zirconia (YSZ-b) particles were pre-distributed into 50 mL tubes. After the urine samples were collected, the urine was added to the 50 mL tubes containing the particles. The borate-passivated yttria-stabilized zirconia (YSZ-b) particles were either allowed to settle to the bottom of the 50 mL tubes by gravity or were centrifuged at 730×g for 10 minutes in a Dynac centrifuge (Clay Adams, a division of Becton, Dickinson, & Company, Parsippany, N.J.). The urine was decanted. The material on the particles was digested with 0.25 U Savinase per mL in a solution containing 4 M guanidine HCl, 1× extraction buffer, and 2.5% (v/v) β-mercaptoethanol (β-ME). The extraction buffer contains 10 mM CAPS (sodium salt (3-[cyclohexylamino]-1-propanesulfonic acid, sodium salt); 10 mM Na₂—CO₃; 10 mM EDTA; 100 mM NaCl; and 0.01% sodium lauroyl sarcosine. Digestion times for samples were generally 1 hour at approximately 55° C. Similar results were obtained for 10 minute digestion times at room temperature (not shown). At the end of the digestion 20×LiCl (20 M LiCl) was added to a final concentration of 1×LiCl (1 M LiCl). For a 1 mL digest, 53 μL of 20×LiCl was added. The material was mixed with gentle vortexing. 70% 2-propanol (Walgreens, Deerfield, Ill.) was added for a total volume of 20 mL with more vortexing.

Samples were either centrifuged at 730×g for 5 minutes or allowed to settle at 1×g through gravity. For optimal PCR results, 2-propanol was allowed to evaporate in a 55° C. drying oven. Samples were considered fully dry when slight cracks started to appear in the surface of the particles. DNA was eluted from all three varieties of particles using 0.5 to 1 mL of 10 mM Tris, pH 8.0. DNA isolated from urine was quantitated with PicoGreen (Molecular Probes, Eugene, Oreg.) using a HindIII digest of lambda (λ) bacteriophage DNA (Sigma-Aldrich, St. Louis, Mo.) as a standard. SigmaPlot (Systat Software Inc, San Jose, Calif.) software was used to fit a linear equation to the relationship of relative fluorescent units as a function of DNA concentration in the standard. The concentration of the standard was determined by the absorbance at 260 nm using an extinction coefficient of 50 μg mL cm⁻¹. The size distribution of DNA fragments isolated from the urine was assessed by electrophoresis using the Lonza (Rockland Me.) FlashGel system with 2.2% agarose gels, 50-1500 base pair DNA ladders, 1× loading dye, and FlashGel camera and software. In some cases, the samples had to be concentrated a second time to be visible on the FlashGels. To concentrate DNA from 1 mL, 10 mM Tris, pH 8.0, 10 μL of particles (OD₄₀₀ₙₘ=50), and 20×LiCl to a final concentration of 1× were added with gentle vortexing. One volume of 100% 2-propanol was added to precipitate the DNA onto the particles. Vortex mixing was for about 20 seconds.

### Example 5

### Nucleic Acid Isolation

The following protocol was generally used to isolate nucleic acids after sample collection. The material on the particles was digested with 0.25 U Savinase per mL in a solution containing 4 M guanidine HCl, 1× extraction buffer, and 2.5% (v/v) β-mercaptoethanol (β-ME). The 20× extraction buffer contains 10 mM CAPS (sodium salt (3-[cyclohexylamino]-1-propanesulfonic acid, sodium salt); 10 mM Na_{z}-CO₃; 10 mM EDTA; 100 mM NaCl; and 0.01% sodium lauroyl sarcosine. Digestion times for samples were generally 1 hour at approximately 55° C. DNA is then isolated by adding 70% isopropanol (2-propanol) to 20 mL. A subsequent rinse step with 10 mL 70% 2-propanol may be added. Excess 2-propanol is evaporated. DNA is eluted with 10 mM Tris, pH 8.0. Other elution buffers may be used as indicated.

### Example 6

### PCR Protocol

The quality of the DNA was assessed using a set of glutathione transferase p (GSTp) multiplex primers. This provided an index for the removal of PCR inhibitors as well as the size range of PCR compatible DNA present in the samples.

The thermocycler conditions were as follows:

1. Hot start at 94′C for 4 minutes;

2. Denature at 94′C for 1 minute;

3. Anneal at 68° C. for 1 minute;

4. Extend at 72′C for 1 minute;

5. Repeated 34 times;

6. Final extension at 72′C for 7 minutes; and

7. Samples held at 5′C.

The GSTp multiplex primers used are shown in Table 2.

TABLE 2

| | | |
|---|---|---|
| SEQ ID NO: 1 | GSTP1C-RP | 5′ - ctc aaa agg ctt cag ttg cc - 3′ |
| SEQ ID NO: 2 | GSTP1C-FP-167 | 5′ - gga gca agc aga gga gaa tc - 3′ |
| SEQ ID NO: 3 | GSTP1C-FP-244 | 5′ - aag gat gga cag gca gaa tg - 3′ |
| SEQ ID NO: 4 | GSTP1C-FP-330 | 5′ - ggc tgt gtc tga atg tga gg - 3′ |
| SEQ ID NO: 5 | GSTP1C-FP-397 | 5′ - cga agg cct tga acc cac t - 3′ |
| SEQ ID NO: 6 | GSTP1C-FP-473 | 5′ - cgt gtg tgt gtg tac gct tg - 3′ |
| SEQ ID NO: 7 | GSTP1C-FP-551 | 5′ - cag aca cag agc aca ttt gg - 3′ |

Roche Diagnostic Fast Tq DNA polymerase was used in addition to standard PCR reactions components at concentrations formulated for this particular enzyme. The PCR reaction products were resolved on 2.2% agarose Flash gels (Lonza, Rockland MW). PCR product fragment size was determined by reference to Lonza DNA FlashGel markers (150 base pairs to 1.5 kilo base pairs). Images were acquired with a Lonza FlashGel camera.

### Example 7

### Comparison of Three Classes of Particles for Isolating DNA from Urine

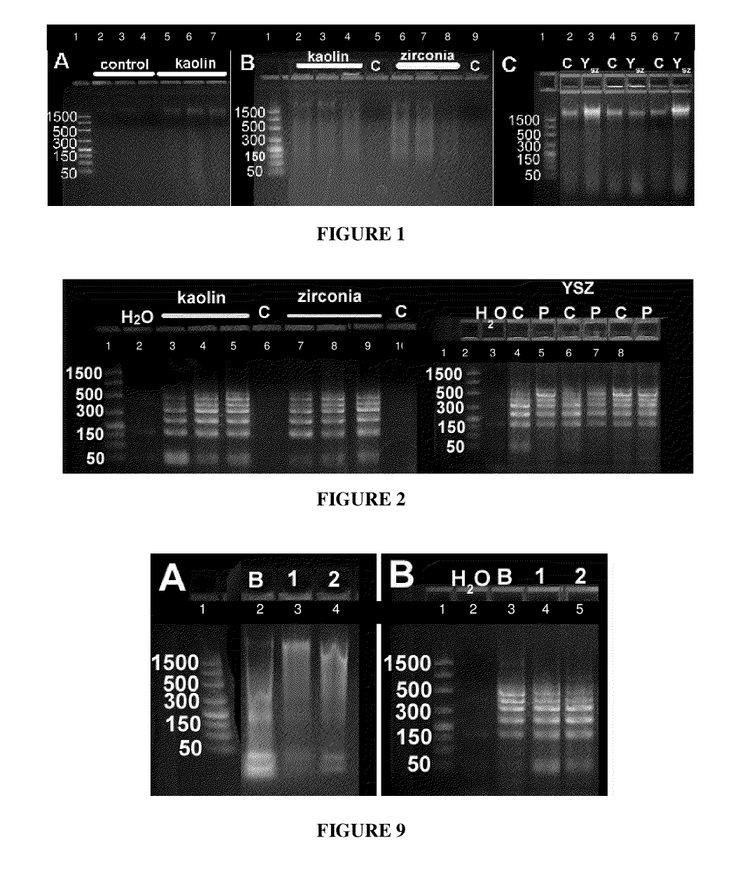

Three types of particles were compared including a mineral, kaolin, a metal oxide, zirconia, and a composite metal oxide, yttria stabilized zirconia. The particles were diluted so the working stock had an absorbance of approximately 50 at 400 nm Particles were then diluted so that the absorbance was less than 1. Urine was collected and split into 50 mL aliquots among six tubes. In FIG. 1A, phosphate/fluoride passivated kaolin particles were added to three of the six samples, (FIG. 1A, lanes 5-7) and three of the six samples contained no particles (FIG. 1A, lanes 2-4). All samples were centrifuged at 730×g centrifugation. Without kaolin, the yield was 83±19 ng DNA per 50 mL of urine, whereas the addition of kaolin increased the yield to 460±141 ng DNA per 50 mL urine. A fragment slightly larger than 1500 base pairs was detected in the samples that were centrifuged with the kaolin particles at 730×g, but this fragment was not detected with the 730×g sedimentation without kaolin as seen in FIG. 1A, lanes 2-4. The kaolin preparation yielded more DNA (p<0.05).

A comparison was also made between phosphate/fluoride passivated kaolin and borate passivated zirconia, as shown in FIG. 1B. A single batch of urine was collected and divided into eight 50 mL tubes. Two of the eight 50 mL tubes received no particles as shown in FIG. 1B, lanes 5 and 9, labeled C for control. Without particles, DNA could be detected by PicoGreen (5 and 20 ng per 50 mL urine) but was not visible on the gel as shown in FIG. 1B, lanes 5 and 9. Kaolin particles yielded 265±70 ng DNA per 1 mL of eluted DNA (or per 50 mL of urine sample), as shown in FIG. 1B, lanes 5-7, whereas the borate-passivated zirconia (ZrO₂-b) particles yielded 200±90 ng DNA per 1 mL of eluted DNA (or per 50 mL of urine sample), as shown in FIG. 1B, lanes 6-8. Comparisons like those in FIG. 1B were made three times using urine samples from three different donors. No significant difference in the DNA yielded was seen between kaolin and borate-passivated zirconia (ZrO₂-b) particles. In replicates not shown, the control amounts of DNA isolated by 730×g centrifugation alone with no particles were within a standard deviation of the mean DNA isolated with the addition of borate-passivated zirconia (ZrO₂-b) particles. In another replicate, DNA isolated in the absence of borate-passivated zirconia (ZrO₂-b) particles was negligible even as measured by PicoGreen (not shown).

A slightly different approach was used in evaluating borate-passivated yttria-stabilized zirconia (YSZ-b). Urine volumes of 300 to 400 mL were split into two samples. Comparisons were made between samples to which no particles were added and samples to which 500 μL of borate-passivated yttria-stabilized zirconia (YSZ-b) particles were added to 200 mL urine. Results are shown in FIG. 1C. In the case of the three comparisons shown in FIG. 1C, the increase in DNA yield using borate-passivated yttria-stabilized zirconia (YSZ-b) (FIG. 1C, lanes 3, 5, and 7) over the controls (FIG. 1C, lanes 2, 4, 6) was 3.4×, 0.8×, and 3.0×. In all three cases, 730×g centrifugation was used to isolate the borate-passivated yttria-stabilized zirconia (YSZ-b) particles. While the amount of cellular debris varied from one collection of urine to another, all three varieties of particles increased the amount of DNA that was isolated from urine centrifuged at 730×g.

### Example 8

### All Three Types of Particles Yield PCR Compatible DNA

The same DNA samples from Example 7 and shown in in FIG. 1 were subjected to GST1p multiplex PCR. The PCR products are shown in FIG. 2. The expected amplicon sizes were 167, 244, 330, 397, 473, and 551 base pairs. The amount of cellular material in the 730×g urine sediment without borate-passivated yttria-stabilized zirconia (YSZ-b) particles was higher in Example 7. PCR products are seen for all samples. Therefore, if there were residual amounts of any of the types of particles in the samples, the particles did not interfere with or inhibit the PCR reactions.

### Example 9

### Borate-Passivated Yttria-Stabilized Zirconia (YSZ-b) Particles Used to Isolate DNA from Urine without a Centrifugation

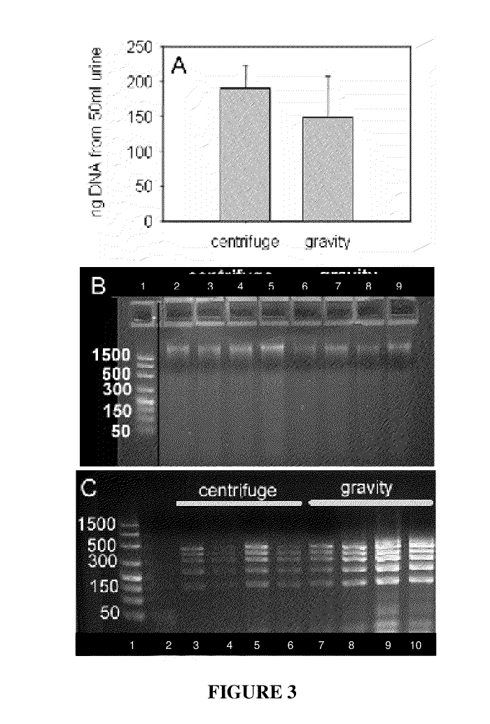

Borate-passivated yttria-stabilized zirconia (YSZ-b) particles were observed to settle faster than the other varieties of particles used. Since gravity is sufficient to cause sedimentation of the borate-passivated yttria-stabilized zirconia (YSZ-b) particles, DNA can be isolated in regions that lack centrifuges and/or the electricity to operate them. A single 400 mL urine collection was split into eight 50 mL aliquots. All aliquots received the same volume of borate-passivated yttria-stabilized zirconia (YSZ-b) particles. The borate-passivated yttria-stabilized zirconia (YSZ-b) particles in the “gravity” samples were allowed to settle for two hours. No significant difference was observed in the amount of DNA isolated with settling through gravity as compared to settling using 730×g centrifugation. As shown in FIG. 3A, the DNA yields were 150±60 ng per 50 mL urine for the gravity settled sample and 190±30 ng per 50 mL urine for the samples settled with centrifugation. The size distribution of the DNA was also very similar for both samples as shown in FIG. 3B. The samples settled with centrifugation are shown in FIG. 3B, lanes 2-5, and the samples settled through gravity alone are shown in FIG. 3B, lanes 6-9. It is noted that there is a DNA fragment slightly larger than 1500 base pair marker and a smaller fragment that appears to extend to less than 50 base pairs. The GST1p multiplex PCR results are shown in FIG. 3C, and the PCR products appear stronger for the gravity isolated samples. The PCR products for the samples settled with centrifugation are shown in FIG. 3C, lanes 3-6, and the PCR products for the samples settled through gravity alone are shown in FIG. 3C, lanes 7-10. It is possible that PCR inhibitors may have co-purified with the DNA when the borate-passivated yttria-stabilized zirconia (YSZ-b) particles were centrifuged at 730×g but not, or to a lesser extent, when the particles were allowed to settle through gravity alone.

### Example 10

### The Influence of Particle Volume on DNA Recovery

This example examines the influence of the volume of particles used to isolate DNA from a sample on the DNA yield. Some collections of urine tend to be visibly cloudy whereas others are very clear. These are inter- and intra-individual variations. There is also some question as to whether the particles facilitate the precipitation of sloughed genital-urinary track epithelial cells or if the particles bind to free DNA.

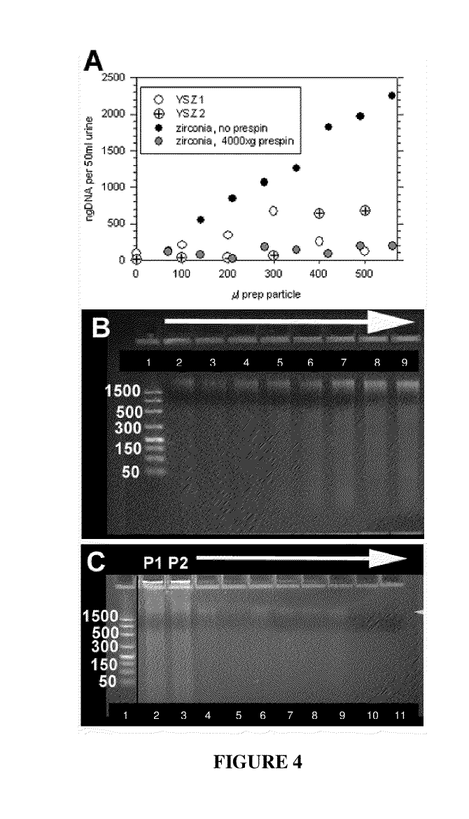

FIG. 4A shows four different urine collections used to examine the influence of the volume of borate-passivated zirconia (ZrO₂-b) and borate-passivated yttria-stabilized zirconia (YSZ-b) particles on the amount and type of DNA isolated. The open circles and the open circles with + signs represent two collections of urine from which DNA was isolated using borate-passivated yttria-stabilized zirconia (YSZ-b) particles. The filled symbols (black circles and gray circles) and represent two separate collections of urine from which DNA was isolated with borate-passivated zirconia (ZrO₂-b) particles. The black circles represent a sample that was processed directly with borate-passivated zirconia (ZrO₂-b) particles, and the gray circles represent a sample that was centrifuged at 4000×g for 15 minutes before processing with borate-passivated zirconia (ZrO₂-b) particles.

For the samples treated with borate-passivated yttria-stabilized zirconia (YSZ-b) particles, the results are shown in Table 3, showing the volume of particles added to each sample and the DNA yield in ng of DNA per 50 mL urine for each sample. These results are also shown in FIG. 4A.

TABLE 3
Borate-passivated yttria-stabilized zirconia (YSZ-b) particles

| | YSZ-b 1 | YSZ-b 2 |
|---|---|---|
| Particle Volume | ng DNA/50 mL urine | ng DNA/50 mL urine |
| 0.00 μL | 91 | 9 |
| 100.00 μL | 205 | 27 |
| 200.00 μL | 205 | 26 |
| 300.00 μL | 670 | 57 |
| 400.00 μL | 248 | 639 |
| 500.00 μL | 118 | 676 |

For the samples treated with borate-passivated zirconia (ZrO₂-b) particles, the results are shown in Table 4, showing the volume of particles added to each sample and the DNA yield in ng of DNA per 50 mL urine for each sample. Data is included for the samples that were not subject to centrifugation and for samples that were subject to centrifugation at 4000 g for 15 minutes. These results are also shown in FIG. 4A.

TABLE 4
Borate-passivated zirconia (ZrO₂-b) particles

| | ZrO₂-b, No Pre-spin | ZrO₂-b, Pre-spin |
|---|---|---|
| Particle Volume | ng DNA/50 mL urine | ng DNA/50 mL urine |
| 70.00 μL | 130 | 107 |
| 140.00 μL | 545 | 77 |
| 210.00 μL | 837 | 14 |
| 280.00 μL | 1063 | 180 |
| 350.00 μL | 1252 | 135 |
| 420.00 μL | 1817 | 91 |
| 490.00 μL | 1962 | 193 |
| 560.00 μL | 2250 | 193 |

FIG. 4B is a photograph of a gel with the DNA isolated from the cloudy urine sample with no pre-centrifugation step using borate-passivated zirconia (ZrO₂-b) particles to isolate the DNA, corresponding to the sample represented by the black circles in FIG. 4A. Note that the large fragment, greater than 1500 base pairs, increases with the increasing volume of borate-passivated zirconia (ZrO₂-b) particles used.

FIG. 4C is a photograph of a gel with the DNA isolated from the urine sample that had been centrifuged at 4000×g for 15 minutes prior to DNA isolation with borate-passivated zirconia (ZrO₂-b) particles, corresponding to the sample represented by the gray circles in FIG. 4A.

In this collection, there was no indication that enough particles had been added to remove all of the DNA in each 50 mL sample of urine. These data also suggest that the particles were causing the sedimentation of DNA that would not sediment with 730×g centrifugation alone. Note that the large fragment, greater than 1500 base pairs, increases with the increasing volume of particles as indicated by the increasing intensity of the band. An increase in smaller DNA fragments pieces is not as apparent.

In another sample shown in FIG. 4A, the urine collection was split between two Sorval GSA bottles and centrifuged at 4000×g for 15 minutes to remove sloughed cells and other large pieces of debris. The pellets (or urine sediment) were digested with Savinase, and DNA was isolated with borate-passivated zirconia (ZrO₂-b) particles, shown by the gray circles in FIG. 4A. FIG. 4C shows the corresponding isolated DNA. Lanes 2 and 3, labeled P1 and P2 in FIG. 4C, show the DNA from the pellets. In FIG. 4C, lanes 4-11 show the DNA isolated from the samples with increasing amounts of with borate-passivated zirconia (ZrO₂-b) particles added, as noted above. In this case, somewhat less than 300 μL of borate-passivated zirconia (ZrO₂-b) particles seemed to be sufficient to remove all of the DNA remaining in the urine after centrifuging at 4000×g, indicated by the plateau in FIG. 4A at a volume of 300 μL of borate-passivated zirconia (ZrO₂-b) particles and greater in isolating DNA from the pre-spun sample.

The arrow in each of FIGS. 4B and 4C indicates an increasing volume of particles. Each lane corresponds to a data point in FIG. 4A. The image in FIG. 4C was digitally enhanced using Adobe Photoshop in order to visualize the DNA. It is interesting to note that both large fragments as well as smaller pieces of degraded DNA were isolated in the fraction that sedimented at 4000×g in the absence of borate-passivated zirconia (ZrO₂-b) particles. This DNA was isolated from the pellets P1 and P2 after Savinase digestion and 2-propanol precipitation onto borate-passivated zirconia (ZrO₂-b) particles.

When using gravity to recover the borate-passivated yttria-stabilized zirconia (YSZ-b) particles and the material adhering to them, 300 μL of particles seems to be an adequate volume for 50 mL urine as shown in FIG. 4A. The decline in yield with increasing volumes of borate-passivated yttria-stabilized zirconia (YSZ-b) particles exceeding 300 μL may be due to a failure to separate eluted DNA from the borate-passivated yttria-stabilized zirconia (YSZ-b) particles without a centrifuge, i.e. a very large void volume. In one unusually clear collection of urine, only as much as 24 ng of DNA were isolated from 50 mL of urine. In this case, the recovered DNA peaked with 300 μL of borate-passivated yttria-stabilized zirconia (YSZ-b) particles (not shown). In another preparation, an increase in DNA yield was seen with 300 μL of borate-passivated yttria-stabilized zirconia (YSZ-b) particles (FIG. 4A). These data do not establish a clearly optimal volume of borate-passivated yttria-stabilized zirconia (YSZ-b) particles or borate-passivated zirconia (ZrO₂-b) particles per volume of urine. These data do suggest that smaller volumes of particles may be sufficient for clear urine that does not contain a high degree of sloughed cells or urine that has been centrifuged to remove sloughed cells.

### Example 11

### The Influence of Time Spent Binding to Borate-Passivated Yttria-Stabilized Zirconia (YSZ-b) Particles on DNA Yield and Quality

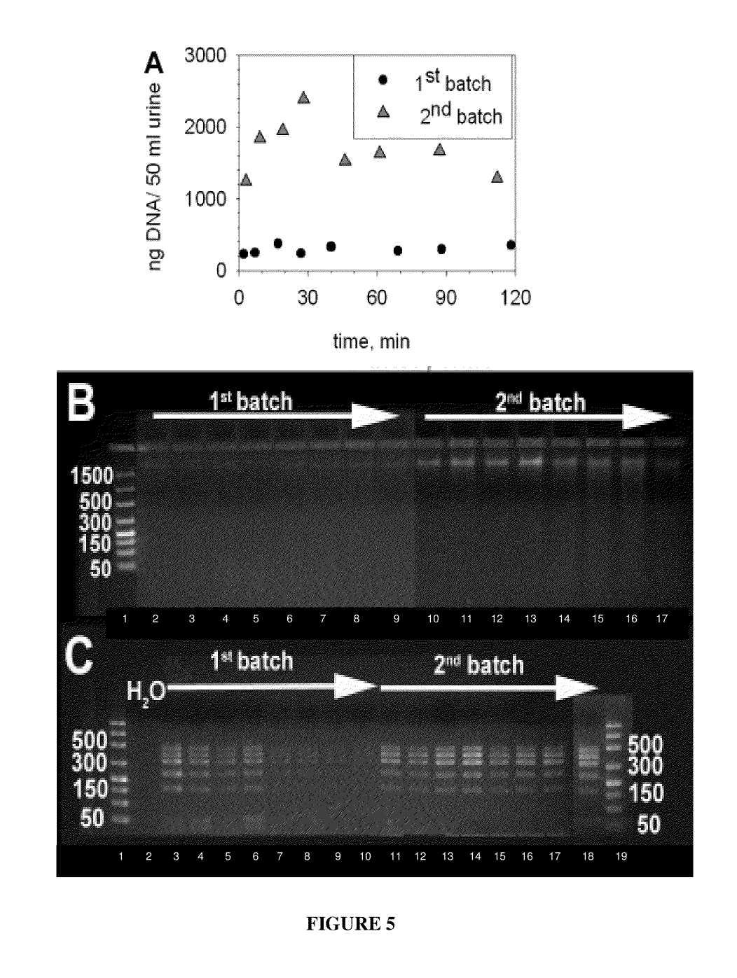

This example illustrates that the about two hours needed for the borate-passivated yttria-stabilized zirconia (YSZ-b) particles to precipitate with gravity is not sufficient time for the nucleases in the urine to degrade a sufficient fraction of the entire amount of DNA in the urine so that it would prevent downstream analysis. In urine from two 400 mL collections, one sample was cloudy and one sample was clear. Each collection was split into eight 50 mL tubes. Borate-passivated yttria-stabilized zirconia (YSZ-b) particles were added to the tubes prior to adding the urine samples. At the times indicated, the tubes were centrifuged at 730×g for 2 minutes with the results shown in Table 5 and FIG. 5A. The second, cloudy batch of urine exhibited more variability in the amounts of DNA isolated at various time points as well as in the intensity of the slightly greater than 1500 base pair fragment (FIG. 5B). Even though the first batch contained less DNA, there was no hint of degradation either as measured by PicoGreen (FIG. 5A), or as seen in a 2.2% agarose gel (FIG. 5B). The region of the gel containing first batch samples had to be digitally enhanced. FIG. 5C shows the PCR products from multiplex PCR using GST1p primers using the DNA shown in FIG. 5B. Arrows indicate increasing time points shown in FIG. 5A. Expected amplicons sizes are 167, 244, 330, 397, 473, and 551 base pairs. Interestingly, there was a decrease in the smaller PCR amplicons in the latter time points in the first batch of urine that was clear (FIG. 5C).

TABLE 5

| 1st batch | | 2nd batch | |
|---|---|---|---|
| Time in Minutes | DNA, ng/mL | Time in Minutes | DNA, ng/mL |
| 2 | 233 | 3 | 1238 |
| 7 | 247 | 9 | 1841 |
| 17 | 376 | 19 | 1948 |
| 27 | 238 | 28 | 2389 |
| 40 | 332 | 46 | 1527 |
| 69 | 275 | 61 | 1629 |
| 88 | 299 | 87 | 1664 |
| 118 | 353 | 112 | 1281 |

### Example 12

### The Ability of Phosphate/Fluoride Treated Kaolin, Borate-Passivated Zirconia (ZrO₂-B), and Borate-Passivated Yttria-Stabilized Zirconia (YSZ-B) Particles to Isolate DNA from a Urine Supernatant

Transrenal DNA (trDNA) is expected to be in the supernatant unless it is associated with the surface of sloughed epithelial cells or heavier debris. Much of the DNA in the urine is hypothesized to be associated with sloughed genital-urinary track cells. If these cells are simply sticking to the particles, then true trDNA from the plasma that gets filtered across the glomeruli might be lost.

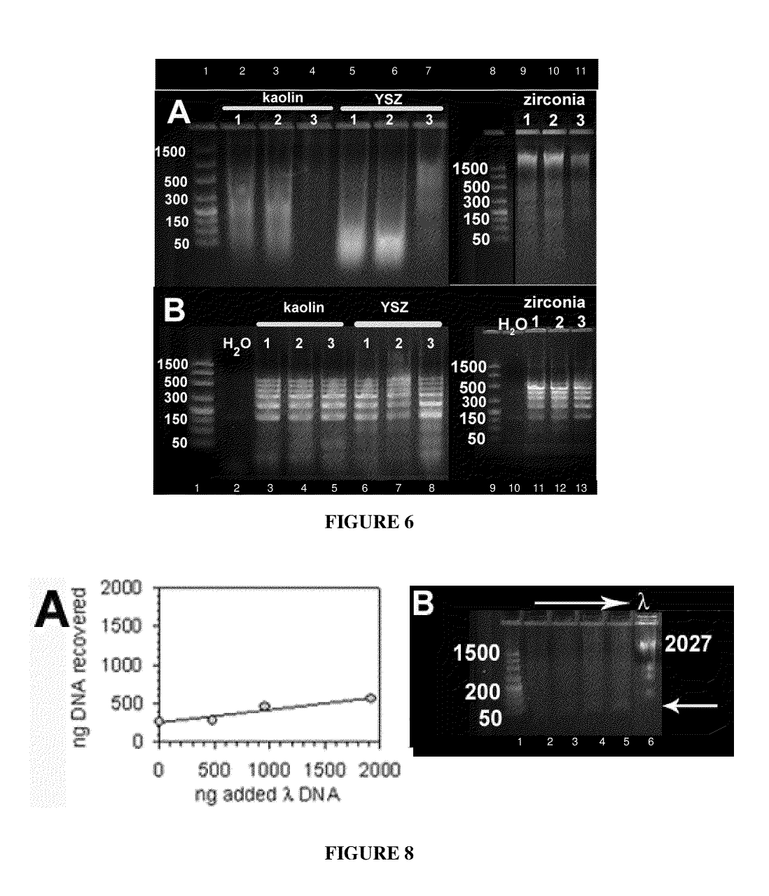

In this example, DNA was isolated from urine using phosphate/fluoride treated kaolin, borate-passivated zirconia (ZrO₂-b), and borate-passivated yttria-stabilized zirconia (YSZ-b) particles. For each of the three particle types, a urine sample was divided in to two aliquots. One aliquot had no particles added (ultimately, fractions 1 and 3) and the other aliquot was incubated with particles (ultimately, fraction 2) as indicated in FIG. 6. Both halves were centrifuged at 730×g for 5 minutes. The pellet from the half that was incubated with particles is referred to as fraction 2. The pellet from the half that did not receive particles is referred to as fraction 1. The supernatant from the half that did not receive particles was mixed with the same volume of particles and centrifuged a second time at 730×g to yield fraction 3.

In these examples, the DNA was isolated from 150 to 200 mL of urine rather than from 50 mL. This was done in order to increase the amount of DNA obtained in fraction 3, that was suspected to contain trDNA. All three fractions were digested with Savinase. After the digestion, particles were added to the digested pellet (fraction 2) from the aliquot that did not receive particles in the first instance. The Savinase digestion was terminated by adding 19 mL of 70% 2-propanol to the 1 mL digest. The particles were rinsed with 10 mL of 70% 2-propanol and evaporated to dryness. DNA was eluted in 10 mM Tris, pH 8.0.

In the kaolin preparation, the vast majority of the DNA was in fractions 1 and 2. Even though there was not enough DNA present in fraction 3 to be visible on a gel, there were sufficient amounts of DNA of sufficient quality to provide a template for all six anticipated PCR products as shown in FIG. 6B. While less DNA was found in fraction 3 in the borate-passivated yttria-stabilized zirconia (YSZ-b) particle preparation, it appeared to be more intact (FIG. 6B). It is possible that nucleases may be found in the 730×g pellets in both the presence and absence of the borate-passivated yttria-stabilized zirconia (YSZ-b) particles. All six expected PCR amplicons were detected with template DNA from all three fractions. A third collection of urine was used to determine if borate-passivated zirconia (ZrO₂-b) particles could remove DNA that remained in the urine after centrifuging at 730×g for 5 minutes. DNA retrieved from the “no particle” supernatant (fraction 3) was largely, if not exclusively greater than 1500 base pairs. DNA from all three borate-passivated zirconia (ZrO₂-b) fractions proved to be good templates for all six expected PCR amplicons (FIG. 6B). The greatest variation appears to be from one collection of urine to another and between individuals. It seems that kaolin particles may be less efficient in retrieving DNA from the urine supernatant. Even if this is the case, the DNA yields the anticipated PCR amplicons without evidence of PCR inhibition.

### Example 13

### Kaolin, Borate-Passivated Zirconia (ZrO₂-b), and Borate-Passivated Yttria-Stabilized Zirconia (YSZ-b) Nanoparticles Eliminate Carryover of PCR Inhibitors Commonly Found with 2-Propanol Nucleic Acid Precipitation

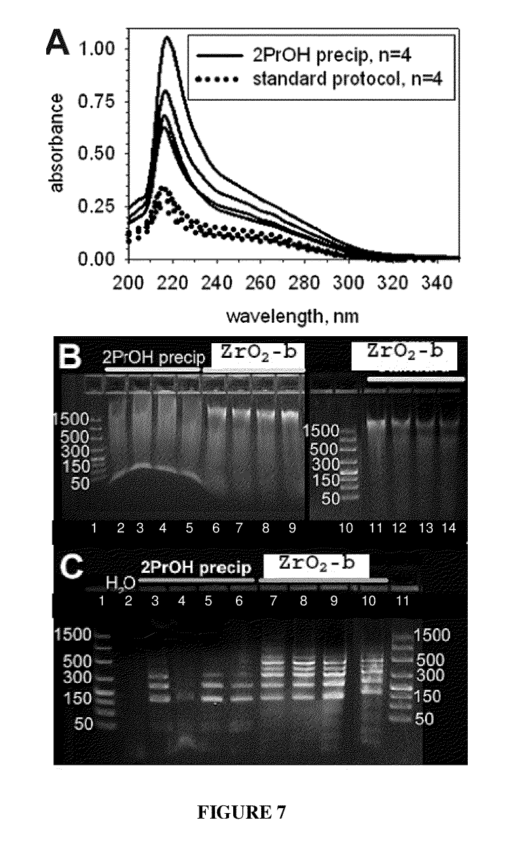

In many nucleic acid isolation methods, 2-propanol is used to precipitate DNA onto high surface area nanoparticles (or lower surface area particles). The nanosurfaces have traditionally proven to be an excellent way of capturing the smaller fragments of DNA. Typically, proteolytic digestions are performed prior to precipitation. Because the protein load in urine was not anticipated to be that great, the 2-propanol precipitation was performed first followed by a digestion with Savinase. DNA from 15 mL of urine was precipitated onto 150 μL of borate-passivated zirconia (ZrO₂-b) particles using two volumes of 70% 2-propanol. For comparison, 50 mL of the same collection of urine was mixed with 500 μL of borate-passivated zirconia (ZrO₂-b) particles. The apparent amount of solids precipitated from 15 mL of urine with 2-propanol and 150 μL of borate-passivated zirconia (ZrO₂-b) particles was greater than the apparent amount of solids precipitated from 50 mL of urine and 500 μL borate-passivated zirconia (ZrO₂-b) particles (not shown). This observation suggests that salts and/or protein may co-precipitate with the 2-propanol. The amount of DNA recovered per mL of urine was the about same in each case, about 60 ng of DNA as measured by PicoGreen. It should be noted that PicoGreen is a specific fluorescent probe for double stranded DNA (dsDNA). UV absorbance spectra for the DNA isolated using either 2-propanol or borate-passivated zirconia (ZrO₂-b) are shown in FIG. 7A with the DNA isolated with 2-propanol shown in black lines and the DNA isolated with borate-passivated zirconia (ZrO₂-b) particles shown in dotted lines. All eight preparations had 260/280 nm ratios of 1.7 or greater. Because uric acid is a derivative of purine metabolism, such high ratios cannot necessarily be considered an index of purity.

These spectra are presented as evidence of the difference between traditional 2-propanol precipitation of nucleic acids and nucleic acid isolation using or borate-passivated zirconia (ZrO₂-b). In this example, DNA was precipitated using 2 volumes of 70% 2-propanol rather than simply centrifuging at 730×g as is Example 5. Attempts to resolve DNA fragments isolated from urine by precipitating with 2-propanol were unsuccessful (FIG. 7B). These data evidence the possible carryover of contaminating materials that interfere with electrophoresis. The DNA isolated from urine using the protocol of Example 5 was rerun without the 2-propanol precipitation samples on the gel (right side of FIG. 7B). Finally, multiplex PCR using primers against GST1p was performed as another comparison between the two DNA preparations. At most, three of the six possible PCR products were detected using DNA that was isolated using a 2-propanol precipitation. In comparison, six of the six possible PCR products were amplified in DNA isolated from the same urine collection using the protocol described in Example 5. (FIG. 7C). The carryover of PCR inhibitors in samples collected by 2-propanol precipitation directly from the urine was anticipated. The similar yields of dsDNA per mL of urine were unanticipated. The DNA isolation protocol used here and in Example 5 can be scaled up to adjust for any volume of urine or other biological sample.

### Example 14

### Failure to Isolate Exogenous DNA from Urine

Nanoparticles, as described herein, can be used to isolate DNA in urine sediment as well as DNA that remains in solution after 4000×g centrifugation as shown in Example 10. This example looks at the isolation of exogenous DNA added to urine. Different sources of DNA were added to determine if exogenous DNA could be recovered from urine using phosphate/fluoride treated kaolin, borate-passivated zirconia (ZrO₂-b), and borate-passivated yttria-stabilized zirconia (YSZ-b) particles as described herein. Three different types of exogenous DNA were added individually to different samples including pooled GST1p PCR products, human and bacterial genomic DNA, and a HindIII digest of lambda (λ) bacteriophage DNA. The results from the use of the HindIII digest of lambda (λ) DNA as the exogenous DNA are shown below in Table 6 and in FIGS. 8A and 8B. The percent recovery shown in Table 6 is calculated as 100× (DNA in spiked sample—endogenous DNA)/exogenous DNA added. The background level of exogenous DNA present in the samples was estimated by measuring the amount of DNA recovered from a sample to which no exogenous lambda (λ) DNA was added. These are the 0 ng λ DNA added rows in Table 6. The DNA concentrations were measured with PicoGreen.

TABLE 6

| | |
|---|---|
| | 2nd batch, shown in |
| 1st batch, not shown | graph and gel |

| λ, DNA | DNA | % | DNA | % |
|---|---|---|---|---|
| added | recovered | recovery | recovered | recovery |
| 0 ng | 58 ng/ml | | 262 ng/ml | |
| 480 ng | 67 ng/ml | 1.9 | 277 ng/ml | 3.1 |
| 960 ng | 74 ng/ml | 1.7 | 454 ng/ml | 20.0 |
| 1920 ng | 87 ng/ml | 1.5 | 565 ng/ml | 15.8 |

Only about 15% of the exogenous linear lambda DNA was recovered with the DNA endogenous to the urine (FIG. 8A). Only the 2027 base pair lambda (λ) HindIII fragment and some degradation products of the original exogenous lambda DNA was visible in this 2.2% agarose gel (FIG. 8B).

DNA from 50 mL of urine and exogenous lambda (λ) DNA was eluted into 0.5 mL Tris, pH 8 and then concentrated a second time to 25 μL. 5 μL of this concentrate was added to the gel shown in FIG. 8B. Significantly, a 50 base pair band is observed in the urine samples to which exogenous lambda (λ) DNA was added. This suggests that lambda DNA was degraded by nucleases in the urine. Lambda (λ) DNA did not bind to any variety of three particle types used in the absence of urine. These data suggest that, in order to survive urine nucleases, DNA may need to be associated with DNA binding proteins. Perhaps the exogenous lambda (λ) DNA was not bound to the necessary proteins. It is possible that DNA binding proteins may be binding to the nanoparticles. The possibility that the urine salts are aiding in the binding by a salting out affect also cannot be excluded.

### Example 15

### The Use of Borate-Passivated Yttria-Stabilized Zirconia (YSZ-b) Particles for Monitoring Chemotherapy

Because borate-passivated yttria-stabilized zirconia (YSZ-b) particles sediment at 1×g with gravity, these nanoparticles can be used to isolate biomolecules from a biological sample without the use of centrifugation. Home urine collections are, therefore, a useful application for borate-passivated yttria-stabilized zirconia (YSZ-b) particles. A volunteer collected urine samples both before and after beginning chemotherapy with erlotinib (Tarceva®) for lung cancer. Erlotinib is an epidermal growth factor receptor (EGFR) inhibitor. If DNA from the tumor cells is filtered into the urine, an increase in the fraction of fragmented DNA may be detected after one and two doses of erlotinib. This was not observed, however, in the analysis on this example as shown in FIG. 9A.

FIG. 9 is a photograph of an agarose gel showing DNA isolated from urine from a chemotherapy patient before and after chemotherapy treatment using borate-passivated yttria-stabilized zirconia (YSZ-b) particles. FIG. 9A shows DNA isolated before treatment (B) and after one (1) and (2) doses of the chemotherapeutic agent erlotinib. FIG. 9B is a photograph of an agarose gel showing the PCR products resulting from multiplexed GST1p PCR using the samples from Example 15 and FIG. 9A. Expected sizes of amplicons are 167, 244, 330, 397, 473, and 551 base pairs.

The origin of the DNA that was isolated is not known. Often chemotherapy agents can slow the growth of normal rapidly dividing cells. These borate-passivated yttria-stabilized zirconia (YSZ-b) particles do offer a useful system for collecting urine DNA over the course of chemotherapy or other therapies that might impact renal function.

### Example 16

### Long Term Storage of DNA

Nanoparticles may also be used for long term storage of DNA and other urine components. In this example, on Day 1, 400 mL of urine was collected, mixed with 2 mL borate-passivated yttria-stabilized zirconia (YSZ-b) particles, and centrifuged at 730×g for 5 minutes, 50 mL at a time in two 50 mL polypropylene tubes. Extra 100 mM Na₂B₄O₇ was added to one tube. Each tube received 5 mL of 70% 2-propanol to expedite the evaporation to dryness at 55° C. Two other batches of urine were collected the next morning. The morning collection was clear, and the evening collection was not. Both were processed the same way. It should be noted that no protease digestion was performed. Samples were capped and allowed to heat continuously at 55° C. until they were digested with Savinase, i.e. by adding the digestion solution to the dry pellet. About 740 ng and 2400 ng of DNA were obtained from the control (with no additional borate added) and extra Na₂B₄O₇ storage from the first day. The samples were processed according to the protocol described in Example 5 except (1) the samples was dried prior to digestion with Savinase, (2) additional borate was added to some of the sample, and (3) the samples were heated for close to two weeks at 55° C. Day 1 samples were heated at 55° C. for 12 days, and Day 2 samples were heated at 55° C. for 11 days before DNA isolation.

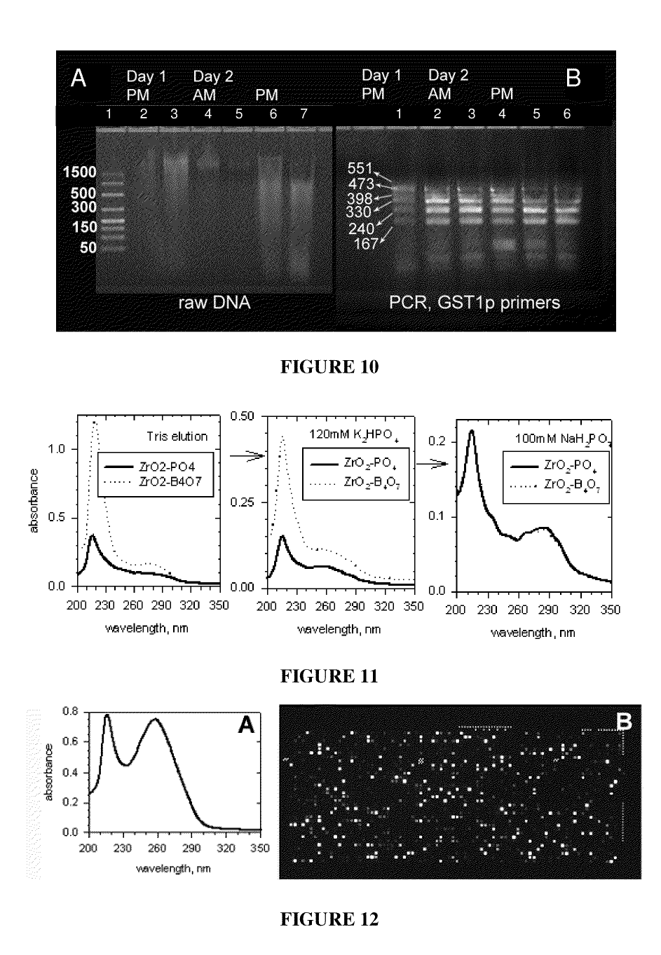

About 750 ng and 370 ng of DNA were obtained from the control and extra Na₂B₄O₇ clear urine from the Day 2 samples. About 3200 and 3500 ng DNA were obtained from the less clear collection of urine from the control and extra Na₂B₄O₇ from the Day 2 samples. These samples were resolved on a 2.2% gel as shown in FIG. 10A. Added Na₂B₄O₇ had been predicted to improve long term storage, but did not appear to in this example. Long term storage at 55° C. was used to simulate the long term storage at 25° C. The Q10 temperature coefficient is a measure of the rate of change of a biological or chemical system as a consequence of increasing the temperature by 10° C.

Q10=(R2/R1)10/(T2−T1)

R2 and R1 are rates of the reaction at temperatures T2 and T1. Q10 is considered to be around 2 for most biological reactions. In other words, the rate is doubled for every 10° C. increase in temperature. A 20° C. increase in temperature would 4× the rate of the reaction. Thus, incubation at 55° C. for two weeks simulates long term storage at 25° C. for about 8 weeks. As shown in FIG. 10B, all expected PRC amplicons are seen in the stored DNA samples indicating that the isolated DNA was preserved in good condition during long-term storage.

### Example 17

### Isolation of RNA from Urine

Nanoparticles can also be used to isolate RNA from urine. Freshly collected clear urine, 100 mL, was mixed with either 1 mL Na₂B₄O₇ passivated zirconia or 1 mL of NaH₂PO₄ passivated zirconia. A Savinase digestion and a 70% 2-propanol precipitation were performed. Excess 2-propanol was evaporated at 55° C. Once the particles were dried, 200 μL of Roche DNAse in the DNAse incubation buffer prepared according to the protocol for the Roche RNA Easy kit was added. The digestion was carried out for 1 hour at 37° C. Extra time was allotted because of the ability of the nanoparticles to isolate very small pieces of dsDNA. The reaction was terminated by the addition of 20 mL of 70% 2-propanol. The material was then centrifuged at 730×g for 5 minutes. The supernatant was discarded. The remaining 2-propanol was evaporated at 55° C. Three sequential elutions were performed. The first (1) with 1 mL 10 mM Tris, pH 8.0; the second (2) with 1 mL 12 mM Na₂HPO₄, pH 8; and the third (3) with 1 mL 50 mM NaH₂PO₄, pH 4.8. The elution profiles are shown in FIG. 11.

RNA concentrations were estimated by RiboGreen (Molecular Probes, Eugene, Oreg.) using mi5-155 as a standard. miR-155 was supplied by Integrated DNA Technologies (San Diego, Calif.). H₂PO₄ passivated zirconia was compared with B₄O₇ passivated zirconia because it has been suggested that vicinal hydroxyl groups in ribonucleic acid may bind to B₄O₇ but not to PO₄ passivation groups. In Example 17, as well as replicates with different urine collections, additional absorption at 255-260 was eluted with 12 mM Na₂HPO₄, pH8. Elution with NaH₂PO₄, pH4.8 phosphate buffer resulted with no evidence of additional elution. Binding of vicinal hydroxyl groups was anticipated to be reversible a low pH. The average extinction coefficient for single-stranded DNA and RNA it is 0.027 (μg/ml)⁻¹ cm⁻¹ and for double-stranded DNA is 0.020 (μg/ml)⁻¹ cm⁻¹. (Sambrook and Russell (2001) Molecular Cloning: A Laboratory Manual , 3rd ed., Cold Spring Harbor Laboratory Press.) The intermediate value of 0.023 (μg/ml)⁻¹ cm⁻¹ was used to estimate the nucleic acid concentration after the absorbance at 350 nm had been subtracted. In Table 7, absorbance readings of the NaH₂PO₄ elutions are considered not applicable (N.A.) since they are so close to base line. It is interesting to note that the RiboGreen data are inconsistent with an approximation of total nucleic acid based on UV absorbance at 260 nm Since elutions were in 1 mL of the indicated solution, mg/mL is the same as mg/100 mL urine.

TABLE 7

| | | Fluorescence | | | |
|---|---|---|---|---|---|
| Elution | | Intensity, | Conc. RNA | UV absorbance | |
| buffer | Particle type | a.u. | (μg/mL) | μg/mL | 260/280 |
| Tris | ZrO₂—PO₄ | 3130 | 1.9 | 4.0 | 1.80 |
| | ZrO₂—B₄O₇ | 3330 | 2.0 | 4.8 | 1.68 |
| Na2HPO4 | ZrO₂—PO₄ | 5200 | 3.2 | 3.0 | 2.00 |
| | ZrO₂—B₄O₇ | 5300 | 3.2 | 4.7 | 1.67 |
| NaH2PO4 | ZrO₂—PO₄ | 965 | 0.5 | N.A. | N.A. |
| | ZrO₂—B₄O₇ | 560 | 0.3 | N.A. | N.A. |

Borate passivation was originally hypothesized to selectively bind to vicinal hydroxyl groups in the ribose backbone of RNA but not in deoxyribose backbone of DNA. The isolation on dilute urine was performed according to the protocol described in Example 5. Phosphate passivated zirconia was prepared according to the protocol for borate passivated zirconia except that NaH₂PO₄ was used as the passivation agent. Three sequential elutions were performed on nucleic acids that had and had not been predigested with DNAse. The first elution was performed with Tris, pH 8 Tris. The second elution was performed with Na₂HPO₄ phosphate buffer, pH about 9, and the third elution was performed with NaH₂PO₄. It was expected that acidic pH may allow for elution of vicinal hydroxyl groups bound to borate groups. The combination of alkali pH and excess phosphate may facilitate the elution of all nucleic acid phosphate groups bound to non-passivated metal oxide groups.

Most interestingly, this example clearly illustrated the isolation of large quantities of RNA from urine.

### Example 18

### Microarray Analysis of RNA Isolated from Urine

Several aliquots of urine were collected (300 mL to 400 mL) to which 1 mL phosphate/fluoride treated kaolin was added. In this example, the OD 400 nm was 320, about six times higher than the standard embodiment of about 50. After each of three collections, the urine was split between two GSA bottles. Samples were centrifuged at 4000×g for 5 minutes. The supernatant was discarded, and the pellets transferred to a −20° C. frost-free freezer. The same GSA bottles were used for serial collections from the same individual using the same batch of kaolin. The previous pellets were only allowed to thaw for as long as it took to centrifuge each additional urine collection. One of the pellets, when it was still frozen, was resuspended in 1 mL of 1× extraction buffer, 4M guanidine HCl, 0.25 mL β-mercaptoethanol, and 100 mL Savinase. The pooled urine samples were digested at 55° C. for one hour. At the end of the digest, 50 mL of 70% 2-propanol was added, as described in Example 5. The sample was centrifuged at 4000×g for 10 minutes in a Sorval GSA rotor. Excess 2-propanol was allowed to evaporate from the pellet at 55° C. for 30 minutes. When slight cracks were visible in the pellet, 10 mL 10 mM Tris, pH 8 was added. The samples were centrifuged in a Sorval GSA rotor for 10 minutes at 4000×g. The supernatant was collected.

miRNAs appear to be important modulators of urologic cancer. MicroRNAs are short RNA molecules (21-23 nucleotides in length) that are found in all eukaryotic cells. miRNAs bind to complementary sequences of messenger RNA (mRNA) resulting in repression of mRNA translation into protein. miRNA expression is frequently altered in tumors, and many are functionally implicated in their pathogenesis. The changes in miRNA spectra observed in the urine samples from patients with different urothelial conditions demonstrates the potential for using concentrations of specific miRNAs in body fluids as biomarkers for detecting and monitoring various physiopathological conditions.

This example demonstrates how nanoparticles are used for isolating RNA from urine. Nucleic acids isolated from urine without additional DNAse treatment for DNA removal were submitted to High Throughput Genomics for micro RNA (miRNA) analysis.

High Throughput Genomics microarray technology is based on a patented quantitative nuclease protection assay (gNPA™) platform. Unlike other gene expression platforms, the qNPA ArrayPlate requires no RNA extraction, cDNA synthesis, RNA amplification, or RNA labeling to be performed. The following, taken from http://www.htgenomics.com/technology/qnpa explains the technology. Specific DNA oligonucleotides are added directly to a Lysis Buffer and hybridize to the RNA present in solution. The DNA oligonucleotides are added in excess to ensure that every molecule of RNA capable of hybridizing to an oligonucleotide does so.

S1 nuclease is added to the hybridized sample buffer. The S1 nuclease is a powerful, single-strand specific nuclease which degrades any non-hybridized (non-double-stranded) nucleic acid. This step effectively removes the non-hybridized portion of the targeted RNA, all of the non-targeted RNA, and excess DNA oligonucleotides.

The S1 nuclease enzyme is completely inactivated. The RNA::DNA hetero-duplexes are then treated to remove the RNA portion of the duplex, leaving only the previously protected oligonucleotide probes.

The resulting DNA oligos are a stoichiometrically representative library of the original RNA sample. The individual DNA probe oligonucleotides are present in the precise relative abundance as the RNA transcripts were in the original sample. The qNPA oligonucleotide library is then ready to be quantified using the ArrayPlate Detection System.

The miRNA-targeted qNPA protection oligos cannot support hybridization of the Detection Linker oligonucleotide due to their short length. Therefore, these qNPA oligonucleotides are biotinylated to facilitate subsequent detection. An avidin-HRP conjugate is used to detect miRNA hybridization instead of the Universal Detection Linker used in the standard protocol.

In the examples given below, expression was normalized to the entire signal. Plant probes were used as negative controls. The signal had to be at least three standard deviations above the negative control to be considered real.

Each element was represented twice on the microarray plate. Microarray analysis was performed twice giving n=4.

One limitation of qNPA technology for miRNA analysis of urine samples is that the RNA concentration should be around 30 to 50 mg/mL. High Throughput Genomics technology is currently applied to formalin fixed paraffin embedded tissue, non-fixed solid tissue, and cultured cells. The concentration of RNA in urine is not high enough to be directly applied to the HTG protocol. The nucleic acids from the urine samples were concentrated first.

After a second purification/concentration step, a 260/280 ratio of 2.0 and an estimated nucleic acid concentration of 32 mg/mL were obtained using the hybrid extinction coefficient described in Example 17. RiboGreen measurements suggest that the concentration is closer to 45 mg/mL. The 260/230 nm ratio in a urine nucleic acid extraction of 1.7 also suggests a pure sample.

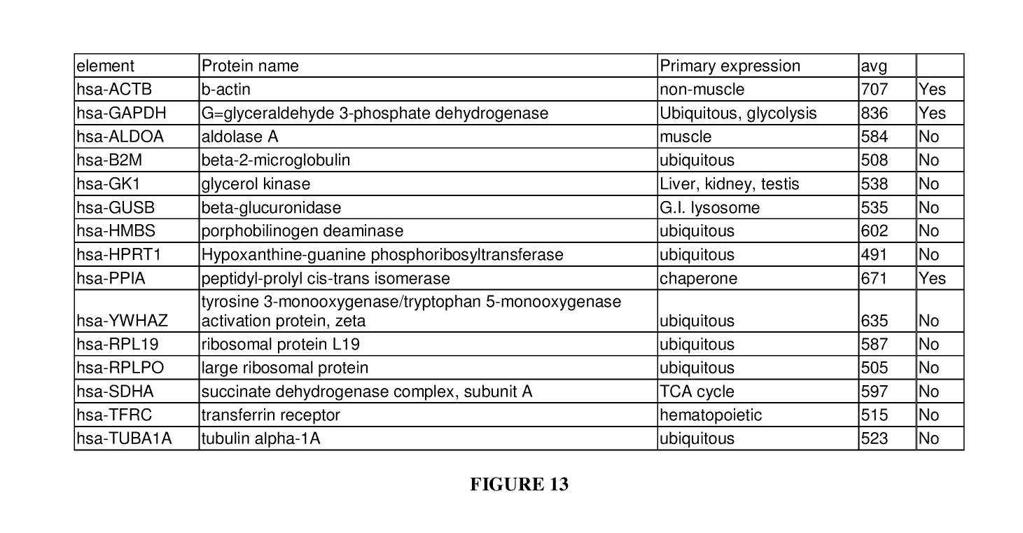

The table in FIG. 13 shows some of the human “house-keeping” genes generally considered to be ubiquitously expressed as well as some with more localized expression. Beta-actin is a highly expressed structural protein in all brush border membranes. GADPH is a kidney house keeping protein. The “avg” column is a quantification of the fluorescent signal for each mRNA listed. The far right hand column containing a “Yes” or “No” for each mRNA indicates whether or not the level of the mRNA found in the sample is determined to be significant. In order to be considered a significant level of expression, the level must be great than three standard deviations above the negative control.

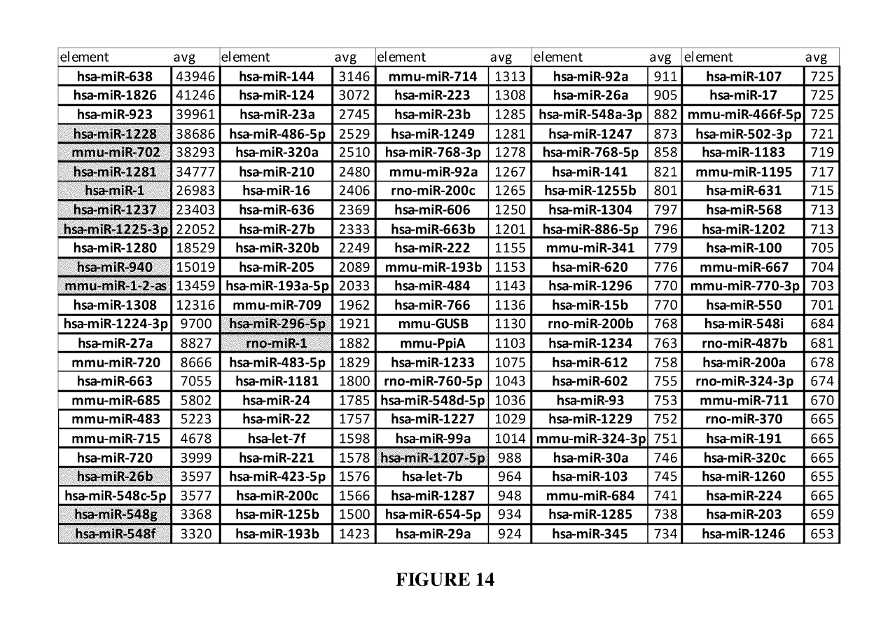

The table in FIG. 14 shows the array of miRNA probes tested that had a significant level of expression greater than three standard deviations above the negative control. The “avg” column is a quantification of the fluorescent signal for each miRNA listed. In order to be considered a significant level of expression, the level must be great than three standard deviations above the negative control. Gray highlighting indicates probes that have high homology to multiple miRNAs and are, therefore, prone to false positive results in spite of having signals greater than three standard deviations above the background.

### Example 19

### Isolation of Proteins from Urine

VeraLight is a medical device company that was established in 2004 to focus on a comprehensive approach to non-invasive type 2 diabetes and pre-diabetes screening with the proprietary SCOUT DS® system. The SCOUT DS® system employs fluorescence spectroscopy to measure advanced glycation end-products (AGEs) in the dermis of an individual's forearm. Veralight's mission is to help stem the tide of the worldwide diabetes epidemic by driving early diabetes detection. Skin AGEs are a well-known biomarker of diabetes, an excellent indicator of cumulative hyperglycemic exposure, and have been shown to predict the development of type 2 diabetes. Two of the most frequently studied skin AGEs are pentosidine, a fluorescent crosslink between lysine and arginine residues, and the lysine derivative, carboxymethyl-lysine (CML). Levels of pentosidine and CML in the skin are positively correlated with the severity of retinopathy, nephropathy and neuropathy. The nanoparticles described herein can be used to isolate proteins from cells and sloughed debris in the urine. The Scout DS® system could potentially be used to detect the same AGEs adhering to nanoparticles prior to digestion with Savinase. Going directly to a primary target of type 2 diabetes, the kidney may enhance the predictive potential of the Scout DS® system.

### Example 20

### Isolation of Protein from Cerebral Spinal Fluid (CSF)

Transmissible spongiform encephalopathies (TSEs) are a group of incurable diseases likely caused by a misfolded form of the prion protein (PrPSc). TSEs include scrapie in sheep, bovine spongiform encephalopathy (“mad cow” disease) in cattle, chronic wasting disease (CWD) in deer and elk, and Creutzfeldt-Jakob disease in humans. Quartz, kaolin, and montmorillonite (Na,Ca)_{0.33}(Al,Mg)₂(Si₄O₁₀)(OH)₂.nH₂O were compared with soil for prion binding capacity. (Johnson C J, PLoS Pathog. 2006 April; 2(4):e32. Epub 2006 Apr. 14.) Montmorillonite was found to have the highest prion binding capacity. CSF may be monitored for TSE associated prions as well as changes in the nucleic acid profile using passivated montmorillonite.

In order to determine if passivated montmorillonite binds prions, passivated and non-passivated montmorillonite can be used on a the same CSF sample.

### Example 21

### Isolation of DNA from Spent Cell Culture Medium

Spent, or used, cell culture medium is expected to contain any soluble material released from necrotic and apoptotic cells. Cell culture medium, like urine, is also high in salts. Unlike urine, it often contains dyes used as pH indicators that do not belong in PCR reactions. DNA isolated from what is released from apoptotic and necrotic cells can be compared with that from the healthy cells in the same cell culture vessel.

### Example 22

### Isolation of RNA from Cerebral Spinal Fluid (CSF)

miRNAs serve as mediators in the brain's response to ischemic preconditioning that leads to endogenous neuroprotection. In addition, microRNAs are implicated in neurodegenerative disorders, including Alzheimer's, Huntington Disease, Parkinson, and Prion disease. The same protocol as described in Example 5 Example 17 can be used to isolate RNA from cerebral spinal fluid (CSF) in order to detect disease markers.

### Example 23

### Kits for Using Nanoparticles for Isolating and Storing Biomolecules from a Biological Sample

Kits for isolating and storing biomolecules from biological samples and instructions for using such kits are provided. For example, kits for carrying out the protocol according to Example 5 are provided.

1. A method of manipulating biomolecules in a biological sample comprising the steps of:
a. providing biological sample;
b. providing a plurality of nanoparticles capable of remaining in a colloidal suspension in an aqueous sample for sufficient time to interact with the biomolecules;
c. incubating the plurality of nanoparticles with the biological sample, wherein the biomolecules become associated with plurality of nanoparticles forming biomolecule-nanoparticle complexes;
d. allowing the biomolecule-nanoparticle complexes to settle out of colloidal suspension from the biological sample;
e. isolating the biomolecule-nanoparticle complexes.

2. The method of claim 1, wherein the biological sample comprises urine, cerebrospinal fluid (CSF), mouthwash samples, or amniotic fluid.

3. The method of claim 1, wherein the biomolecule comprises nucleic acids, proteins, cells, cell fragments, bacteria, or viruses.

4. The method of claim 1 wherein the biological sample comprises urine and the biomolecule comprises DNA.

5. The method of claim 1, wherein the biological sample comprises urine and the biomolecule comprises RNA.

6. The method of claim 1, wherein the biological sample comprises urine and the biomolecule comprises protein.

7. The method of claim 1, wherein the plurality of nanoparticles comprise borate-passivated yttria-stabilized zirconium oxide.

8. The method of claim 1, wherein the plurality of nanoparticles comprise borate-passivated yttria-stabilized zirconium oxide.

9. The method of claim 4, wherein the plurality of nanoparticles comprise borate-passivated yttria-stabilized zirconium oxide.

10. The method of claim 4, wherein the plurality of nanoparticles comprise borate-passivated zirconium oxide.

11. The method of claim 1, further comprising storing the biomolecule-nanoparticle complexes.

12. The method of claim 11, further comprising eluting the biomolecule from the biomolecule-nanoparticle complexes.

13. A method of isolating nucleic acids from urine comprising the steps of:
a. providing a urine sample;
b. providing a plurality of nanoparticles capable of remaining in a colloidal suspension in an aqueous sample for sufficient time to interact with the biomolecules;
c. incubating the plurality of nanoparticles with the urine sample, wherein the biomolecules become associated with plurality of nanoparticles, forming biomolecule-nanoparticle complexes;
d. allowing the biomolecule-nanoparticle complexes to settle out of colloidal suspension from the urine;
e. isolating the biomolecule-nanoparticle complexes.

14. The method of claim 13, further comprising storing the biomolecule-nanoparticle complexes.

15. The method of claim 13, further comprising eluting the biomolecules from the biomolecule-nanoparticle complexes.

16. The method of claim 13, wherein the plurality of nanoparticles comprise borate-passivated yttria-stabilized zirconium oxide.

17. The method of claim 16, wherein the plurality of nanoparticles are allowed to settle out of colloidal suspension without centrifugation.

18. The method of claim 13, wherein the plurality of nanoparticles comprise borate-passivated zirconium oxide.

19. A kit for isolating and storing biomolecules from a biological sample comprising:
a. a vessel containing a plurality of nanoparticles capable of remaining in a colloidal suspension in an aqueous sample for sufficient time to interact with the biomolecules;
b. instructions for collecting a biological sample and for incubating the biological sample with the plurality of nanoparticles to form biomolecule-nanoparticle complexes;

20. The kit according to claim 20 wherein the plurality of nanoparticles comprise borate-passivated yttria-stabilized zirconium oxide.
